(19) United States
(12) Patent Application Publication
(10) Pub. No.: US 2026/0041866 A1
(43) Pub. Date: Feb. 12, 2026

(54) APPARATUS AND METHODS FOR ENDOTRACHEAL TUBE SECUREMENT AND ADJUSTMENT

(71) Applicant: BOARD OF REGENTS, THE UNIVERSITY OF TEXAS SYSTEM, Austin, TX (US)

(72) Inventors: Daniel STROMBERG, Austin, TX (US); Sung Hoon JUNG, Austin, TX (US); Chloe IHONVBERE, Austin, TX (US); Rose ANCONETANI, Austin, TX (US); Scott DAVENPORT, Austin, TX (US); Justine LE, Austin, TX (US)

(21) Appl. No.: 19/104,302

(22) PCT Filed: Aug. 21, 2023
(86) PCT No.: PCT/US2023/072562

§ 371 Date: Feb. 17, 2025

Related U.S. Application Data
(60) Provisional application No. 63/399,802, filed on Aug. 22, 2022.
(60) Provisional application No. 63/426,261, filed on Nov. 17, 2022.

(51) Int. Cl. A61M 16/04

(52) U.S. Cl. CPC A61M 16/0497; A61M 16/0461; A61M 2205/581; A61M 2205/582

(57) ABSTRACT

Exemplary embodiments of the present disclosure relate to apparatus and methods for securely positioning an endotracheal tube within a patient's airway and precisely adjusting the positioning of the endotracheal tube. In particular embodiments, an apparatus comprises a tubular member configured to receive an endotracheal tube, a housing comprising an opening configured to receive the tubular member, a first engagement mechanism coupled to the housing, and a second engagement mechanism coupled to the housing.

### CROSS-REFERENCE TO RELATED APPLICATIONS

This application claims priority to U.S. Provisional Patent Application Ser. No. 63/399,802 filed Aug. 22, 2022 and U.S. Provisional Patent Application Ser. No. 63/426,261 filed Nov. 17, 2022, the entire contents of each of which are incorporated herein by reference.

### BACKGROUND INFORMATION

Long term patients, including intensive care unit (ICU) patients, often require prolonged mechanical ventilation via an endotracheal tube (ETT). In order to properly function, these tubes must reside in the trachea at the proper depth, and thus they are typically secured to the patient's lip and facial structures using tape to prevent migration of the ETT. Patient movement, oral and/or nasal secretions, and adhesive deterioration can lead to improper positioning of the ETT. Accordingly, readjustment and retaping of the ETT are frequently required, often on a daily basis.

However, attempts at adjustment up or down are commonly undertaken with respect to physical patient landmarks (e.g. the gum line) without benefit. For example, an order may be placed to pull the ETT out 0.5 centimeters if too deep, and after adjustment and repeat chest x-ray, the position has not changed. Repetition of the procedure is then required with all the attendant risks (frequently including re-sedation of the patient), repeat x-ray exposure, and cost inefficiencies of duplicative care delivery. The procedural lack of success can sometimes be attributed to the imprecise nature of estimating where tube gradations line up with patient landmarks.

In addition, patients can be made unstable during ETT repositioning, and accidental removal of the ETT from the trachea can be fatal to the patient. Accordingly, ETT repositioning is stressful to the medical personnel, which can cause such personnel to rush their efforts to get the ETT resecured as quickly as possible and lead to potential further complications.

Accordingly, a need exists for apparatus and methods to address the shortcomings of existing techniques used for ETT securement and adjustment in a patient's airway.

### SUMMARY

Exemplary embodiments of the present disclosure relate to apparatus and methods for securely positioning an endotracheal tube within a patient's airway and precisely adjusting the positioning of the endotracheal tube.

Exemplary embodiments include an apparatus comprising: a tubular member configured to receive an endotracheal tube; a housing comprising an opening configured to receive the tubular member; a first engagement mechanism coupled to the housing; and a second engagement mechanism coupled to the housing; where: the first engagement mechanism is configured to be placed in either an unlocked position or a locked position; the second engagement mechanism is configured to be placed in either an unlocked position or a locked position; the tubular member can be moved in a first direction and a second direction within the opening in the housing when the first engagement mechanism and the second engagement mechanism are both in the unlocked position; the tubular member can only be moved in the first direction within the opening in the housing when the first engagement mechanism is in the unlocked position and the second engagement mechanism is in the locked position; the tubular member can only be moved in the second direction within the opening in the housing when the second engagement mechanism is in the unlocked position and the first engagement mechanism is in the locked position; and the tubular member cannot be moved in either the first direction or the second direction within the opening in the housing when the first engagement mechanism and the second engagement mechanism are both in the locked position.

In particular embodiments the tubular member is a two-piece tubular member comprising a first piece and a second piece, wherein the second piece is configured to secure to the first piece along a longitudinal axis. In certain embodiments the tubular member comprises: a first end, a second end, a first set of serrated teeth and a second set of serrated teeth; the first set of serrated teeth comprises angled surfaces angled toward the first end; and the second set of serrated teeth comprises angled surfaces angled toward the second end. In some embodiments the first engagement mechanism comprises a first spring-loaded engagement member configured to engage the first set of serrated teeth; and the second engagement mechanism comprises a second spring-loaded engagement member configured to engage the second set of serrated teeth.

In particular embodiments, the first engagement mechanism comprises a first push button configured to rotate a first annular cam such that the first engagement mechanism can be moved from the locked position to the unlocked position and from the unlocked position to the locked position by depressing the first push button; and the second engagement mechanism comprises a second push button configured to rotate a second annular cam such that the second engagement mechanism can be moved from the locked position to the unlocked position and from the unlocked position to the locked position by depressing the second push button. In certain embodiments, the first engagement mechanism further comprises: a first barrel with a first plurality of grooves; and

a first rotating cam with a first plurality of ridges, where the first plurality of ridges is slidably engaged with the first plurality of grooves; and the second engagement mechanism further comprises: a second barrel with a second plurality of grooves; and a second rotating cam with a second plurality of ridges, where the second plurality of ridges are slidably engaged with the second plurality of grooves.

In specific embodiments the first engagement mechanism comprises a first rotatable cap configured to rotate such that the first engagement mechanism can be moved from the locked position to the unlocked position and from the unlocked position to the locked position; and the second engagement mechanism comprises a second rotatable cap configured to rotate such that the second engagement mechanism can be moved from the locked position to the unlocked position and from the unlocked position to the locked position.

In certain embodiments the first engagement mechanism comprises: a first spring engaging the first spring-loaded engagement member; and a second spring engaging the first spring-loaded engagement member; and the second engagement mechanism comprises: a third spring engaging the second spring-loaded engagement member; and a fourth spring engaging the second spring-loaded engagement member. In particular embodiments the first spring exerts a force on the first spring-loaded engagement member toward the first set of serrated teeth; the second spring exerts a force on the first spring-loaded engagement member away from the first set of serrated teeth; the third spring exerts a force on the second spring-loaded engagement member toward the second set of serrated teeth; and the fourth spring exerts a force on the second spring-loaded engagement member away from the second set of serrated teeth.

In some embodiments the first spring-loaded engagement member engages the first set of serrated teeth when the first engagement mechanism is in the locked position; the first spring-loaded engagement member does not engage the first set of serrated teeth when the first engagement mechanism is in the unlocked position; the second spring-loaded engagement member engages the second set of serrated teeth when the second engagement mechanism is in the unlocked position; and the second spring-loaded engagement member does not engage the second set of serrated teeth when the second engagement mechanism is in the unlocked position. In specific embodiments movement of the tubular member within the opening in the housing in a first direction compresses the first spring when the first engagement mechanism is in the locked position; and movement of the tubular member within the opening in the housing in a second direction compresses the third spring when the second engagement mechanism is in the locked position.

In certain embodiments the first engagement mechanism comprises a first locking member positioned between the first spring and the first rotatable cap; and the second engagement mechanism comprises a second locking member positioned between the third spring and the second rotatable cap. In particular embodiments the first engagement mechanism can be moved from the unlocked position to the locked position by compressing the first spring with the first locking member and rotating the first rotatable cap; and the second engagement mechanism can be moved from the unlocked position to the locked position by compressing the third spring with the second locking member and rotating the second rotatable cap.

In specific embodiments the first rotatable cap comprises a first aperture; the second rotatable cap comprises a second aperture; the first locking member is configured to extend through the first aperture when the first engagement mechanism is in the unlocked position; the second locking member is configured to extend through the second aperture when the second engagement mechanism is in the unlocked position; the first locking member is configured to be contained within the first rotatable cap when the first engagement mechanism is in the locked position; and the second locking member is configured to be contained within the second rotatable cap when the second engagement mechanism is in the locked position. In some embodiments the housing comprises a slot configured to receive an endotracheal tube stabilization device.

In certain embodiments the first spring-loaded engagement member comprises a first planar member configured to engage the first spring and the second spring; and the second spring-loaded engagement member comprises a second planar member configured to engage the third spring and the fourth spring. In particular embodiments the first spring-loaded engagement member comprises a first extension extending from the first planar member; the first extension comprises a first angled end configured to engage the first set of serrated teeth; the second spring-loaded engagement member comprises a second extension extending from second the planar member; and the second extension comprises a second angled end configured to engage the second set of serrated teeth. In specific embodiments the first extension extends through the second spring; and the second extension extends through the fourth spring.

Certain embodiments include a method of adjusting an endotracheal tube, the method comprising: coupling an apparatus to an endotracheal tube stabilization device, wherein the apparatus comprises: a housing comprising an opening configured to receive the tubular member and the endotracheal tube; a first engagement mechanism coupled to the housing, wherein the first engagement mechanism is configured to be placed in either an unlocked position or a locked position; a second engagement mechanism coupled to the housing, wherein the second engagement mechanism is configured to be placed in either an unlocked position or a locked position; coupling a tubular member to an endotracheal tube; inserting the tubular member and the endotracheal tube into the opening of the housing; moving the first engagement mechanism from the unlocked position to the locked position; adjusting the tubular member and the endotracheal tube within the opening in the housing; and moving the second engagement mechanism from the unlocked position to the locked position, such that the tubular member and the endotracheal tube are restricted from further movement within the opening in the housing.

In particular embodiments adjusting the tubular member and the endotracheal tube within the opening in the housing provides a precise endotracheal tube position adjustment within an airway of a patient. In some embodiments moving the first engagement mechanism from the unlocked position to the locked position and moving the second engagement mechanism from the unlocked position to the locked position holds the endotracheal tube in a stable position in the airway of the patient. In specific embodiments the first engagement mechanism comprises a first rotatable cap, a first spring, a second spring and a first locking member; the second engagement mechanism comprises a second rotatable cap, a third spring, a fourth spring, and a second locking member; moving the first engagement mechanism from the unlocked position to the locked position comprises: compressing the first spring by depressing the first locking member within the first rotatable cap; and rotating the first rotatable cap; and moving the second engagement mechanism from the unlocked position to the locked position comprises: compressing the third spring by depressing the second locking member within the second rotatable cap; and rotating the second rotatable cap.

In some embodiments, the first engagement mechanism comprises a first push button, a first spring, a second spring and a first locking member; the second engagement mechanism comprises a second push button, a third spring, a fourth spring, and a second locking member; moving the first engagement mechanism from the unlocked position to the locked position comprises compressing the first spring by depressing the first push button; and moving the second engagement mechanism from the unlocked position to the locked position comprises compressing the third spring by depressing the second push button.

In particular embodiments, the first engagement mechanism further comprises: a first barrel with a first plurality of grooves; and a first rotating cam with a first plurality of ridges, wherein the first plurality of ridges are slidably engaged with the first plurality of grooves; the second engagement mechanism further comprises: a second barrel with a second plurality of grooves; and a second rotating cam with a second plurality of ridges, wherein the second plurality of ridges are slidably engaged with the second plurality of grooves.

In specific embodiments the tubular member comprises a first set of serrated teeth and a second set of serrated teeth; the first engagement mechanism comprises a first spring-loaded engagement member; the second engagement mechanism comprises a second spring-loaded engagement member; moving the first engagement mechanism from the unlocked position to the locked position causes the first spring-loaded member to engage the first set of serrated teeth; and moving the second engagement mechanism from the unlocked position to the locked position causes the second spring-loaded member to engage the second set of serrated teeth. In some embodiments adjusting the tubular member and the endotracheal tube in a first direction within the opening in the housing provides an audible feedback to a user when the first spring-loaded engagement member is engaged with the first set of serrated teeth.

In certain embodiments adjusting the tubular member and the endotracheal tube in a second direction within the opening in the housing provides an audible feedback to a user when the second spring-loaded engagement member is engaged with the second set of serrated teeth. In particular embodiments adjusting the tubular member and the endotracheal tube in a first direction within the opening in the housing provides a haptic feedback to a user when the first spring-loaded engagement member is engaged with the first set of serrated teeth. In specific embodiments adjusting the tubular member and the endotracheal tube in a second direction within the opening in the housing provides a haptic feedback to a user when the second spring-loaded engagement member is engaged with the second set of serrated teeth.

In the present disclosure, the term “coupled” is defined as connected, although not necessarily directly, and not necessarily mechanically.

The use of the word “a” or “an” when used in conjunction with the term “comprising” in the claims and/or the specification may mean “one,” but it is also consistent with the meaning of “one or more” or “at least one.” The terms “approximately, “about” or “substantially” mean, in general, the stated value plus or minus 10%. The use of the term “or” in the claims is used to mean “and/or” unless explicitly indicated to refer to alternatives only or the alternative are mutually exclusive, although the disclosure supports a definition that refers to only alternatives and “and/or.”

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises,” “has,” “includes” or “contains” one or more steps or elements, possesses those one or more steps or elements, but is not limited to possessing only those one or more elements. Likewise, a step of a method or an element of a device that “comprises,” “has,” “includes” or “contains” one or more features, possesses those one or more features, but is not limited to possessing only those one or more features. Furthermore, a device or structure that is configured in a certain way is configured in at least that way, but may also be configured in ways that are not listed.

As used herein the terms “first conduit” and “second conduit” includes both conduits that are separate from each other, as well as first and second portions of a continuous conduit. For example, a single conduit coupled to a peristaltic pump coupled can comprise a “first conduit” at inlet side of the pump and a “second conduit” at the outlet side of the pump.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will be apparent to those skilled in the art from this detailed description.

### BRIEF DESCRIPTION OF THE FIGURES

The following drawings form part of the present specification and are included to further demonstrate certain aspects of the present invention. The invention may be better understood by reference to one or more of these drawings in combination with the detailed description of specific embodiments presented herein.

FIGS. 1-4 illustrate perspective views of an apparatus according to an exemplary embodiment of the present disclosure during different stages of assembly.

FIG. 5 illustrates a perspective view of the housing of the embodiment of FIGS. 1-4.

FIG. 6 illustrates perspective views of an endotracheal tube and of a tubular member of the embodiment of FIGS. 1-4.

FIG. 7 illustrates bottom view of the tubular member of FIG. 6.

FIG. 8 illustrates perspective and end views of the tubular member of FIG. 6.

FIG. 9 illustrates an exploded view of a first engagement mechanism of the embodiment of FIGS. 1-4.

FIG. 10 illustrates section and elevation view of the first engagement mechanism of FIG. 9 in an unlocked position.

FIG. 11 illustrates section and elevation view of the first engagement mechanism of FIG. 9 in a locked position.

FIG. 12 illustrates an exploded view of a second engagement mechanism of the embodiment of FIGS. 1-4.

FIG. 13 illustrates section and elevation view of the second engagement mechanism of FIG. 12 in an unlocked position.

FIG. 14 illustrates section and elevation view of the second engagement mechanism of FIG. 12 in a locked position.

FIG. 15 illustrates a first spring-loaded engagement member engaged with a first set of serrated teeth of the embodiment of FIGS. 1-4 in different positions.

FIG. 16 illustrates a first perspective view of an apparatus according to the to an exemplary embodiment of the present disclosure.

FIG. 17 illustrates a second perspective view of the embodiment of FIG. 16.

FIG. 18 illustrates an exploded view of an engagement mechanism according to an exemplary embodiment of the present disclosure.

FIG. 19 illustrates a partial section view of the embodiment of FIG. 18 in a disengaged position.

FIG. 20 illustrates a partial section view of the embodiment of FIG. 18 in an engaged position.

FIG. 21-24 illustrate partial side section views of the embodiment of FIG. 18 in different positions during operation.

### DETAILED DESCRIPTION OF ILLUSTRATIVE EMBODIMENTS

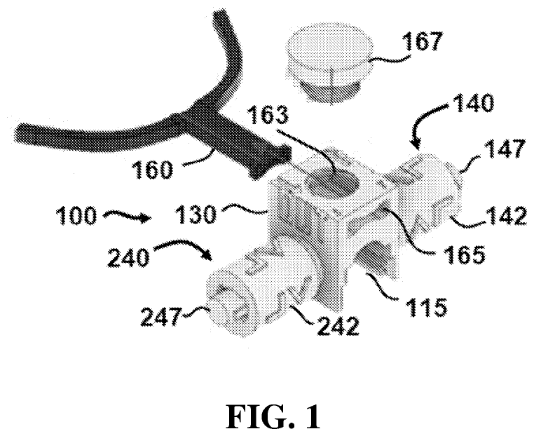

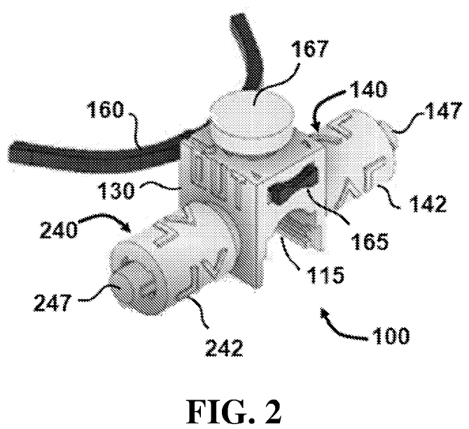

For purposes of clarity, certain elements in some of the figures may not be labeled with a reference number. Referring initially to FIGS. 1-5, an apparatus 100 includes a housing 130 comprising an opening 115 configured to receive tubular member 110, which is in turn configured to receive an endotracheal tube 120. In the embodiment shown, housing 130 also comprises a slot 165 configured to receive an endotracheal tube stabilization device 160. In certain embodiments, housing 130 comprises an aperture 163 configured to receive a retaining member 167 to retain and secure endotracheal tube stabilization device 160. In particular embodiments, aperture 163 is a threaded hole and retaining member 167 is a threaded member (e.g. a thumbscrew or other configuration capable of being installed and removed without the use of external tools). The illustrated embodiment also includes a first engagement mechanism 140 a second engagement mechanism 240, each coupled to housing 130.

In the embodiment shown, housing 130 comprises a removable base 166 (shown in

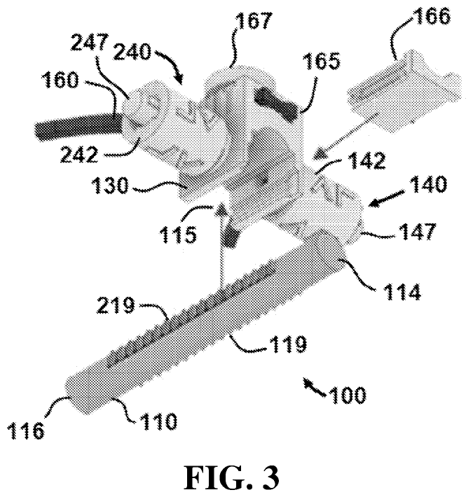

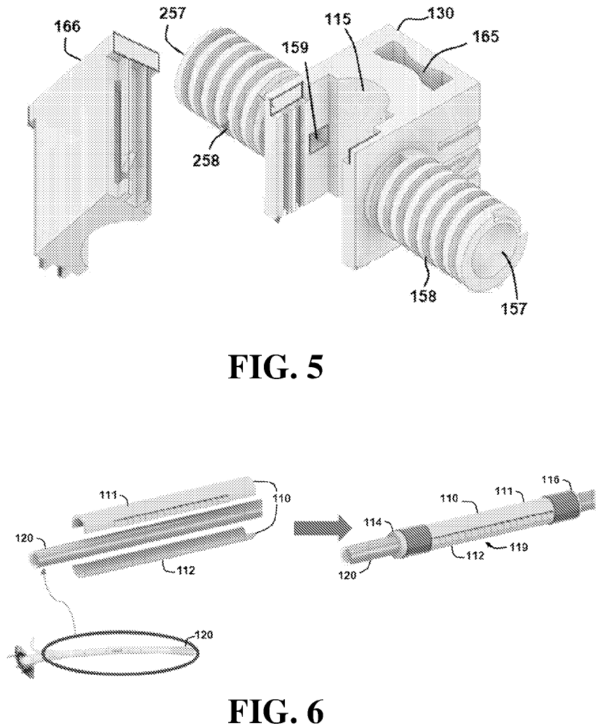

FIGS. 3-5) that retains tubular member 110 within opening 115. In addition, housing 130 comprises first cylindrical extension 158 comprising an aperture 157, and a second cylindrical extension 258 comprising an aperture 257 (shown in FIG. 5). First engagement mechanism 140 is configured to couple to first cylindrical extension 158, while second engagement mechanism 240 is configured to couple to second cylindrical extension 258.

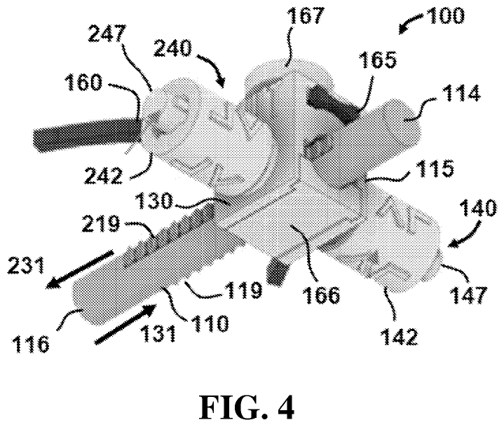

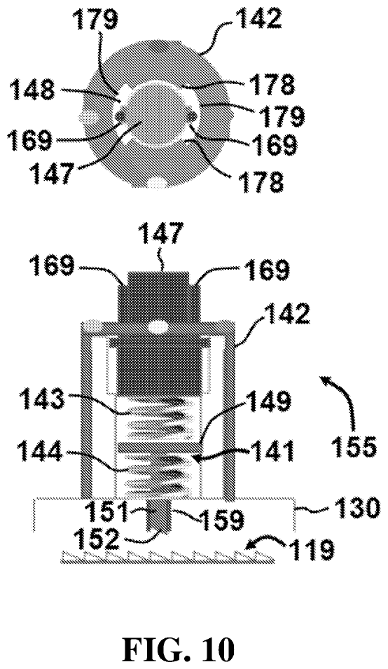

Both first engagement mechanism 140 and second engagement mechanism 240 are configured to be placed in either an unlocked position or a locked position, as shown in FIGS. 10-14. As explained further below, tubular member 110 (and an endotracheal tube inserted within tubular member 110) can be moved in a first direction 131 and a second direction 231 (shown in FIG. 4) within an opening 115 in housing 130 when first engagement mechanism 140 and the second engagement mechanism 240 are both in the unlocked position.

However, tubular member 110 can only be moved in first direction 131 within opening 115 in housing 130 when first engagement mechanism 140 is in the unlocked position and second engagement mechanism 240 is in the locked position. Similarly, tubular member 110 can only be moved in second direction 231 within opening 115 in housing 130 when second engagement mechanism 240 is in the unlocked position and first engagement mechanism 140 is in the locked position. Finally, tubular member 110 cannot be moved in either first direction 131 or second direction 231 within opening 115 in housing 130 when first engagement mechanism 140 and second engagement mechanism 240 are both in the locked position. This configuration allows a user to secure tubular member 110 and endotracheal tube 120 within a patient's airway and then precisely position tubular member 110 and endotracheal tube 120 within the airway.

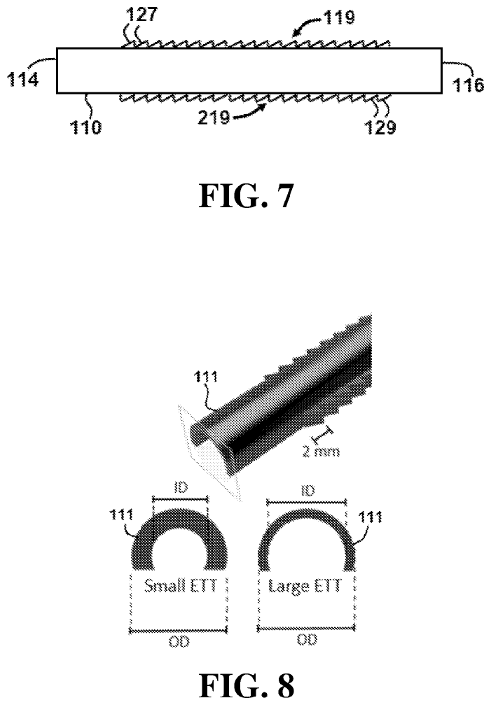

Referring now to FIGS. 6-7, are exploded and bottom views of one embodiment of tubular member 110, respectively. In this embodiment, tubular member 110 is shown as a two-piece tubular member comprising a first piece 111 and a second piece 112, wherein the second piece is configured to secure to the first piece along a longitudinal axis 113. In this embodiment, first piece 111 of tubular member 110 comprises a first end 114, a second end 116, a first set of serrated teeth 119 and a second set of serrated teeth 219. In the embodiment shown, first set of serrated teeth 119 comprises angled surfaces 127 angled toward first end 114 and second set of serrated teeth 219 comprises angled surfaces 129 angled toward second end 116. In particular embodiments, first and second sets of serrated teeth 119 and 219 may comprise individual teeth that are approximately 2.0 millimeters long (e.g. measured in the direction of longitudinal axis 113) and 1.0 millimeters high (e.g. measured perpendicular to longitudinal axis 113. While the embodiment shown portrays serrated teeth extending outwardly from the outer diameter of tubular member 110, it is understood that other embodiments may comprise serrated teeth that extend inwardly (e.g. toward the center of tubular member 110).

In certain embodiments, first piece 111 and second piece 112 can be coupled together via an adhesive tape, snap fit, or other suitable coupling arrangements. FIG. 8 illustrates different embodiments of tubular member first piece 111 comprising different size inner diameters configured to receive different sizes of endotracheal tubes. In the embodiments shown in FIG. 8 tubular members 110 maintain a consistent outer diameter (e.g. 7.25 mm in one embodiment) so that tubular members with different inner diameters can be utilized with the same housing 130.

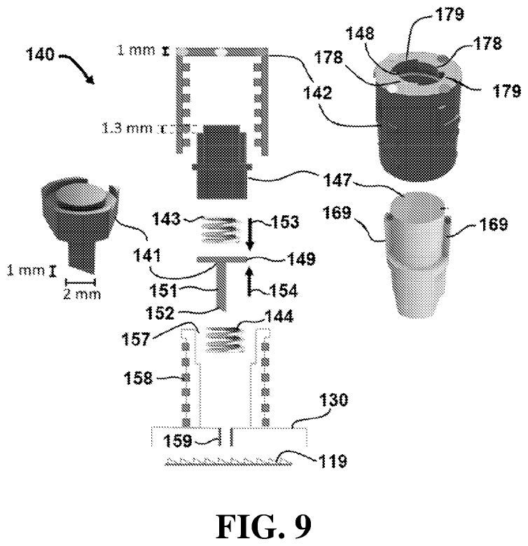

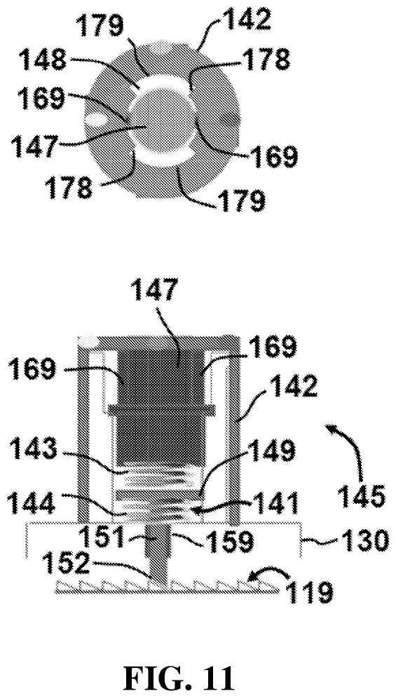

FIG. 9 illustrates an exploded view of first engagement mechanism 140. It is understood that second engagement mechanism 240 is equivalent to first engagement mechanism 140, with minor orientation exceptions as noted further below. In the embodiment shown, first engagement mechanism 140 comprises a first spring-loaded engagement member 141 configured to engage the first set of serrated teeth 119. In the illustrated embodiment, first engagement mechanism 140 comprises a first rotatable cap 142 configured to rotate such that the first engagement mechanism 140 can be moved from a locked position 145 (shown in FIG. 11) to an unlocked position 155 (shown in FIG. 10) and from the unlocked position 155 to the locked position 145.

In the embodiment shown in FIG. 9, first engagement mechanism 140 comprises a first spring 143 and a second spring 144, each engaging first spring-loaded engagement member 141. In this embodiment, first spring 143 exerts a force 153 on first spring-loaded engagement member 141 toward first set of serrated teeth 119, and second spring 144 exerts a force 154 on first spring-loaded engagement member 141 away from first set of serrated teeth 119.

As shown in FIGS. 10-11, first spring-loaded engagement member 141 engages first set of serrated teeth 119 when first engagement mechanism 140 is in the locked position 145, and first spring-loaded engagement member 141 does not engage first set of serrated teeth 119 when first engagement mechanism 140 is in the unlocked position 155. Movement of tubular member 110 within opening 115 in housing 130 in first direction 131 (shown in FIG. 4) compresses first spring 143 when engagement mechanism 140 is in locked position 145 (shown in FIG. 11).

In the embodiment shown, first engagement mechanism 140 comprises a first locking member 147 positioned between first spring 143 and first rotatable cap 142. In the illustrated embodiment, first spring-loaded engagement member 141 comprises a first planar member 149 configured to engage first spring 143 and second spring 144. First spring-loaded engagement member 141 also comprises a first extension 151 extending from the first planar member 149 and through second spring 144 in the embodiment shown. In addition, first extension 151 comprises a first angled end 152 configured to extend through aperture 159 and engage the first set of serrated teeth 119. First engagement mechanism 140 can be moved from the unlocked position 155 to the locked position 145 by compressing the first spring 143 with the first locking member 147 and rotating the first rotatable cap 142, as explained below.

In the embodiment shown, first rotatable cap 142 comprises a first aperture 148 configured to allow locking member 147 to extend through cap 142 or be retained within cap 142 depending on the rotational position of cap 142. In the illustrated embodiment, aperture 148 comprises a larger circumference portion 179 configured to allow extensions 169 of locking member 147 to pass through cap 142 when cap 142 is rotated to unlocked position 155 to align larger circumference portion 179 with extensions 169. In addition, aperture 148 comprises a smaller circumference portion 178 configured to retain extensions 169 within cap 142 when cap 142 is rotated to locked position 145 to align smaller circumference portion 178 with extensions 169. Accordingly, when first engagement mechanism 140 is in unlocked position 155, a user can align larger circumference portion 179 with extensions 169 and press down on first locking member 147. The user can then rotate first cap 142 so that smaller circumference portion 178 is aligned with extensions 169. This allows first cap 142 to retain first locking member 147, compressing first spring 143 and engaging first spring-loaded engagement member 141 with first set of serrated teeth 119 in locked position 145.

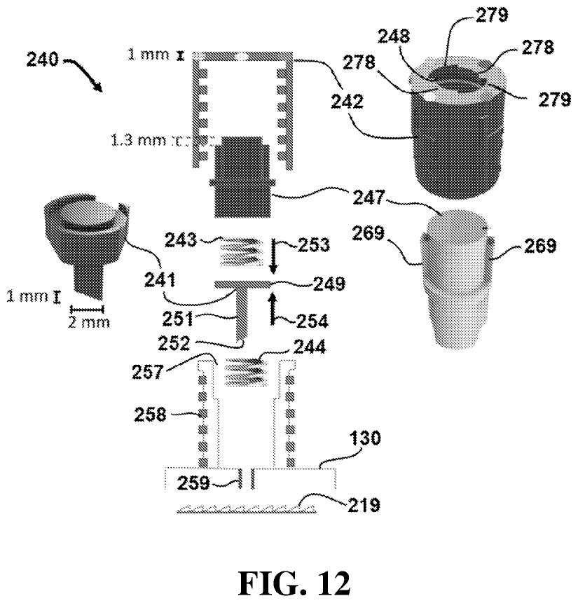

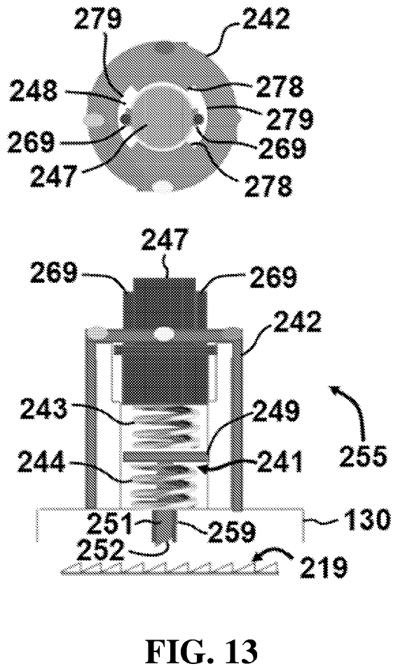

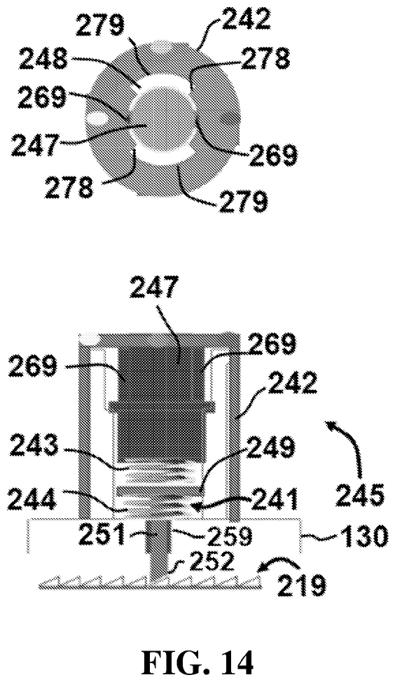

FIG. 12 illustrates an exploded view of second engagement mechanism 240. The components in second engagement mechanism 240 are equivalent to those of first engagement mechanism 140, but can be oriented differently as discussed below. In the embodiment shown, second engagement mechanism 240 comprises a second spring-loaded engagement member 241 configured to engage the second set of serrated teeth 219. In the illustrated embodiment, second engagement mechanism 240 comprises a second rotatable cap 242 configured to rotate such that the second engagement mechanism 240 can be moved from a locked position 245 (shown in FIG. 14) to an unlocked position 255 (shown in FIG. 13) and from the unlocked position 255 to the locked position 245.

In the embodiment shown in FIG. 12, second engagement mechanism 240 comprises a third spring 243 and a fourth spring 244, each engaging second spring-loaded engagement member 241. In this embodiment, third spring 243 exerts a force 253 on second spring-loaded engagement member 241 toward second set of serrated teeth 219, and fourth spring 244 exerts a force 254 on second spring-loaded engagement member 241 away from second set of serrated teeth 219.

As shown in FIGS. 13-14, second spring-loaded engagement member 241 engages second set of serrated teeth 219 when second engagement mechanism 240 is in the locked position 245, and second spring-loaded engagement member 241 does not engage second set of serrated teeth 219 when second engagement mechanism 240 is in the unlocked position 255. Movement of tubular member 120 within opening 115 in housing 130 in second direction 231 (shown in FIG. 4) compresses third spring 243 when engagement mechanism 240 is in locked position 245 (shown in FIG. 14).

In the embodiment shown, second engagement mechanism 240 comprises a second locking member 247 positioned between third spring 243 and second rotatable cap 242. In the illustrated embodiment, second spring-loaded engagement member 241 comprises a second planar member 249 configured to engage third spring 243 and fourth spring 244. Second spring-loaded engagement member 241 also comprises a second extension 251 extending from the second planar member 249 and through second spring 244 in the embodiment shown. In addition, second extension 251 comprises a second angled end 252 configured to extend through aperture 259 and engage the second set of serrated teeth 219.

The second engagement mechanism 240 can be moved from the unlocked position 255 to the locked position 245 by compressing the third spring 243 with the second locking member 247 and rotating the second rotatable cap 242 in a manner equivalent to the previously described first engagement mechanism 140. In the embodiment shown, second rotatable cap 242 comprises a second aperture 248 configured to allow second locking member 247 to extend through cap 242 or be retained within cap 242 depending on the rotational position of cap 242. In the illustrated embodiment, aperture 248 comprises a larger circumference portion 279 configured to allow extensions 269 of locking member 247 to pass through cap 242 when cap 242 is rotated to unlocked position 255 to align larger circumference portion 279 with extensions 269. In addition, aperture 248 comprises a smaller circumference portion 278 configured to retain extensions 269 within cap 242 when cap 242 is rotated to locked position 245 to align smaller circumference portion 278 with extensions 269.

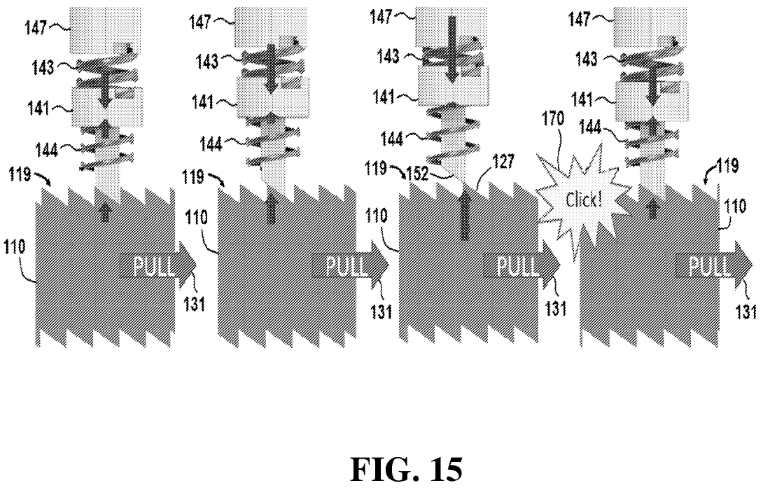

Embodiments of the present disclosure allow a user to precisely adjust an endotracheal tube within a patient's airway. Referring now to FIG. 15, first spring-loaded engagement member 141 is shown engaged with first set of serrated teeth 119 as tubular member 110 is moved in direction 131. In the embodiment shown, as tubular member 110 is moved in direction 110, first set of serrated teeth 119 initially move first spring-loaded engagement member 141 toward first locking member 147, compressing first spring 143. This movement of first spring-loaded engagement member 141 is shown in the left view and two central views of FIG. 15. As tubular member 110 is moved in direction 131, first angled end 152 is directed toward first locking member by angled surface 127. After first tubular member 110 has been moved a sufficient distance in direct 131 (e.g. equivalent to the length of one of serrated teeth 119, which in certain embodiments is 2.0 millimeters), first spring 143 moves first spring-loaded engagement member 141 away from first locking member 147 and toward tubular member 110. This movement provides an audible and haptic feedback 170 to the user in the form of a clicking sound and feel. Audible and haptic feedback 170 allows a user to precisely position and adjust an endotracheal tube within a patient's airway.

Similar adjustments can be made in direction 231 (shown in FIG. 4) with second locking member 247 and second set of serrated teeth 219. Accordingly, a user can initially place tubular member 110 and an endotracheal tube 120 within a patient's airway by coupling apparatus 100 to an endotracheal tube stabilization device 160 affixed to the patient. In exemplary embodiments of the present disclosure. endotracheal tube 120 can be either an oro-tracheal tube or a naso-tracheal tube. Accordingly, apparatus 100 can be configured to insert endotracheal tube 120 via oral or nasal passages. In embodiments configured for use with a naso-tracheal tubes, endotracheal tube stabilization device 160 may comprise extensions or arms that are shorter and/or more angled than those shown in FIGS. 1-4. Apparatus 100 can initially be configured such that first engagement mechanism 140 and second engagement mechanism 240 are both in the unlocked position. In one embodiment, the user can then move second engagement mechanism 240 to the locked position and further insert tubular member 110 in direction 231. The engagement of second spring-loaded engagement member 241 (not visible in FIG. 4) with second set of serrated teeth 219 provides haptic and audible feedback to indicate to the user how far tubular member 110 has been inserted. If the user is satisfied with the positioning of tubular member, first engagement mechanism 140 can then be moved to the locked position, securing tubular member 110 and endotracheal tube 120 in a fixed location in the patient's airway. If the user determines that further adjustment is needed, the user can move either first engagement mechanism 140 or second engagement mechanism 240, to retract or further insert tubular member 110, respectively. When the user has determined that tubular member 110 is in the desired location with the airway, both first engagement mechanism 140 and second engagement mechanism 240 can be placed in the locked position, restricted from further movement of tubular member 110 and endotracheal tube 120 within the opening 115 in housing 130 of apparatus 100. Accordingly, tubular member 110 and endotracheal tube 120 are secured from further insertion or retraction within the patient's airway.

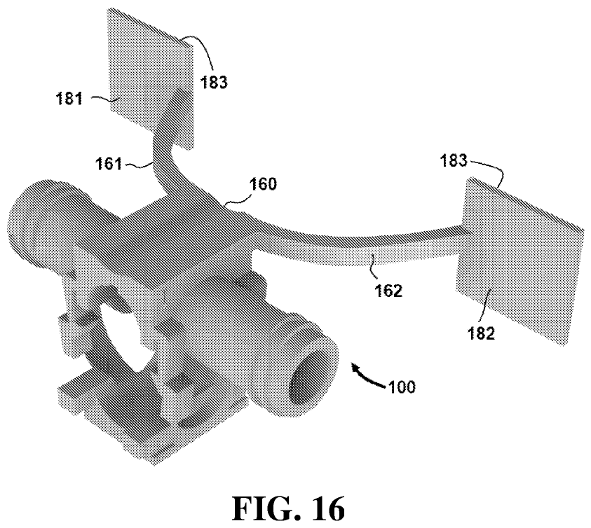

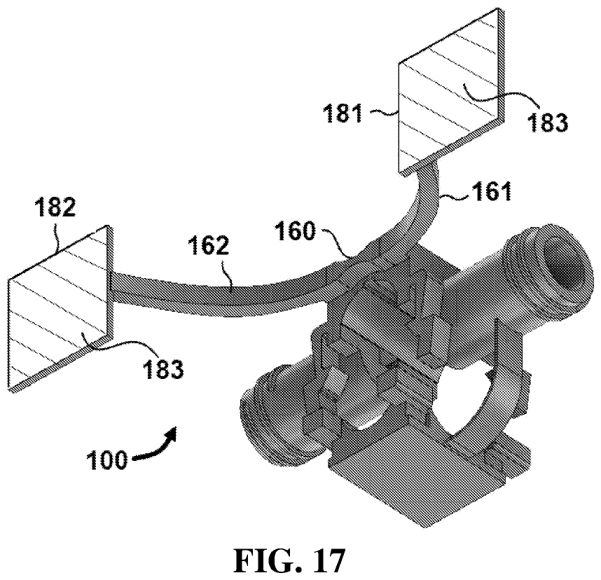

Referring now to FIGS. 16 and 17, perspective views of an embodiment of apparatus 100 are shown with an endotracheal tube stabilization device 160 that includes extensions 161 and 162 respectively coupled to pads 181 and 182 configured to engage the face of the patient. As used herein, the terms “pad” and “pads” are interpreted to include generally planar members that distribute forces over the surface area of the member. The terms do not require (but do not exclude) any form of cushioning or deformable aspect. In particular embodiments, pads 181 and 182 may comprise an adhesive 183 that can adhere to the skin of the patient and assist in stabilization of the apparatus 100.

In aspects other than endotracheal tube stabilization device 160, apparatus 100 shown in FIGS. 16-17 is generally equivalent to the embodiment previously described and shown in FIGS. 1-15. The description of elements shown and described in FIGS. 1-15 is not repeated here for sake of brevity, but it is understood that the embodiment shown in FIGS. 16-17 includes such features.

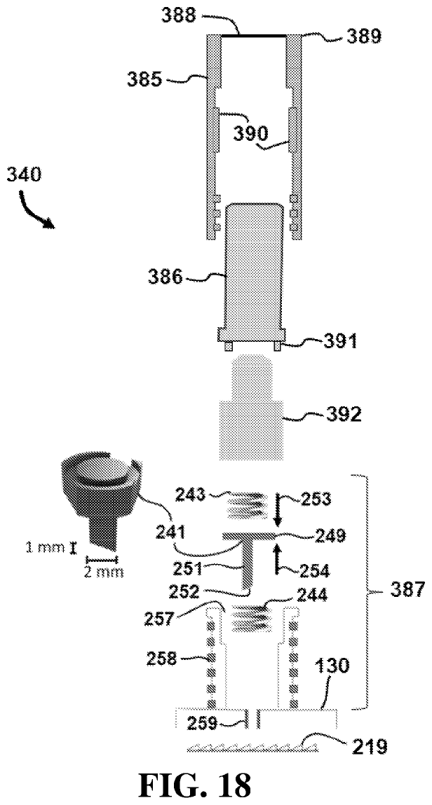

Referring initially to FIG. 18, another embodiment of an engagement mechanism 340 is illustrated comprising a push button mechanism. In the embodiment shown in FIG. 18, components that are equivalent to previously-described embodiments are labeled with reference numbers identical to those in previously-described embodiments. The operation and functionality of such components will not be repeated here for sake of brevity, but one skilled in the art will understand the equivalently numbered components will operate in a manner as discussed in the previously-described embodiments. It is also understood that engagement mechanism 340 can be utilized in previously described embodiments in lieu of first and/or second engagement mechanisms 140 and 240. For example, other embodiments of apparatus 100 shown in FIGS. 1-5 may comprise engagement mechanisms equivalent to engagement mechanism 340 instead of engagement mechanisms 140 and 240. In still other embodiments of apparatus 100, only one engagement mechanism is utilized and the tubular member includes only one set of serrated teeth.

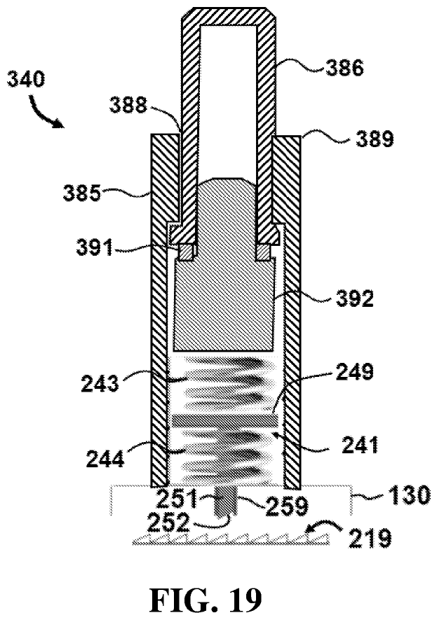

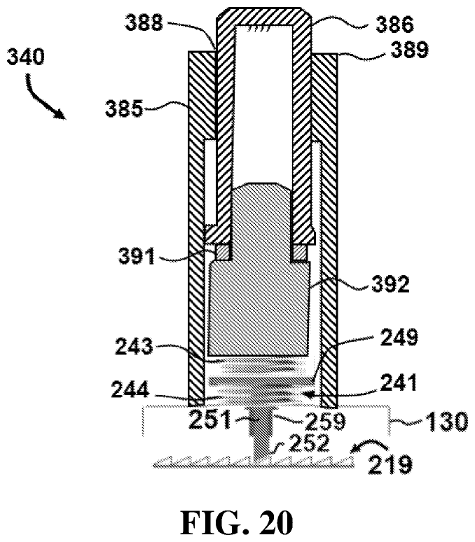

FIG. 18 illustrates an exploded view of engagement mechanism 340, while FIGS. 19 and 20 illustrate partial section view of engagement mechanism 340 in disengaged and engaged positions, respectively. Aspects of engagement mechanism 340 are similar to push button mechanisms shown and described in U.S. Pat. No. 6,062,756, incorporated by reference herein. In the illustrated embodiment, engagement mechanism 340 comprises a barrel 385 encompassing a push button 386, a rotating cam 392 and a lower assembly 387 comprising components as discussed in the embodiments shown and described in FIGS. 9-12. In this embodiment, barrel 385 is threaded onto cylindrical extension 258 and push button 386 extends through an aperture 388 in a distal end 389 of barrel 385. Barrel 385 further comprises a grooved cam 390, while push button 386 comprises an annular cam 391 configured to engage rotating cam 392.

During operation, springs 243 and 244 exert a force on rotating cam 392 and push button 386 such that push button 386 is initially fully extended through aperture 388 as shown in FIG. 19 in a disengaged position. An operator can change the position of engagement mechanism 340 from the disengaged position shown in FIG. 19 to an engaged position shown in FIG. 20 by depressing push button 386 (e.g. exerting a force on push button 386 to compress springs 243 and 244). As explained further below, depressing push button 386 can cause engagement member 241 to engage or disengage with serrated teeth 219.

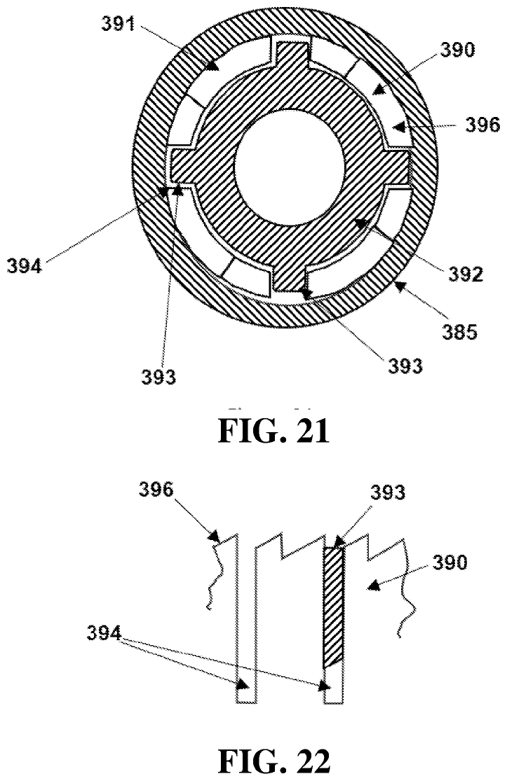

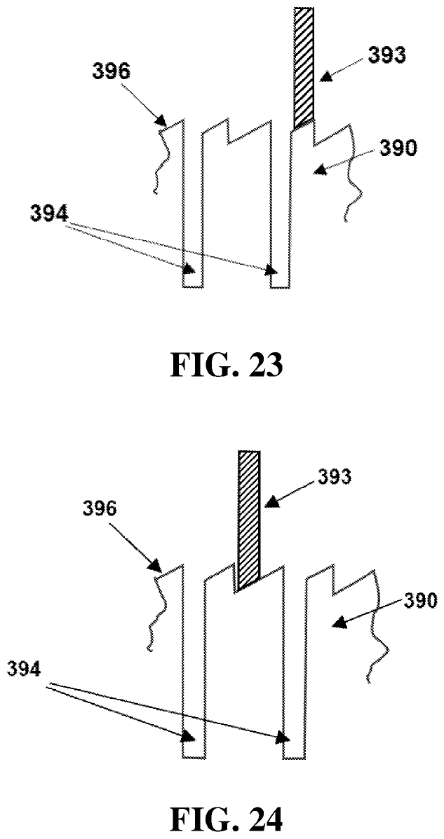

FIGS. 21-24 show the relation between rotating cam 392 and grooved cam 390 in barrel 385 during operation. FIG. 21 provides a partial end section view, while FIGS. 22-24 provide partial side section views. In this embodiment, rotating cam 392 comprises several longitudinal ridges 393 on its outer surface. In operation, longitudinal ridges 393 are slidably inserted in longitudinal grooves 394 of barrel 385. A sawtooth cam 396 is formed in the front end of the grooved cam 390 of barrel 385. Annular cam 391 of push button 386 is formed by arranging a plurality of triangular projections in a circle. Rotating cam 392 advances or rotates when the push button 386 is pushed forward in a state wherein engagement member 241 is retracted behind the open front end of barrel 385 as shown in FIG. 19, and the ridges of rotating cam 392 are fitted in longitudinal grooves of grooved cam 390 of barrel 385 as shown in FIG. 22. Annular cam 391 of push button 386 exerts a force on the rotating cam 392 in a circumferential direction, causing the ridges of rotating cam 392 to be displaced in the same circumferential direction. When the pressure is removed from push button 386 (e.g. the user no longer exerts a force on push button 386), the ridges of rotating cam 392 come into engagement with sawtooth cam 396 in barrel 385 as shown in FIG. 23. Accordingly, engagement member 241 is projected from the open front of barrel 385 as shown in FIG. 20 by the pushing action of rotating cam 392 and is held in the engaged position as shown in FIG. 24. Longitudinal ridges 393 are moved forward away from sawtooth cam 396 in barrel 385 and are displaced in the circumferential direction when push button 386 is pushed again with engagement member 241 projected forward, longitudinal ridges 393 of the rotating cam 392 engage with the next longitudinal grooves 394 of barrel 385 when the pressure is then removed from push button 386, and longitudinal ridges 393 of the rotating cam 392 slide backward along longitudinal grooves 394 of barrel 385 to a position shown in FIG. 19 by the force of springs 243 and 244 in lower assembly 387.

### * * * * * *

All of the apparatus, systems and/or methods disclosed and claimed herein can be made and executed without undue experimentation in light of the present disclosure. While the devices, systems and methods of this invention have been described in terms of particular embodiments, it will be apparent to those of skill in the art that variations may be applied to the devices, systems and/or methods in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### References

The contents of the following references are incorporated by reference herein:
U.S. Pat. No. 6,062,756
US20110108038
US20140261462
US20210038846
US20210213224
CN206837215
CN216022565
WO20110091

1. An apparatus comprising:
a tubular member configured to receive an endotracheal tube;
a housing comprising an opening configured to receive the tubular member;
a first engagement mechanism coupled to the housing; and
a second engagement mechanism coupled to the housing;
wherein:
the first engagement mechanism is configured to be placed in either an unlocked position or a locked position;
the second engagement mechanism is configured to be placed in either an unlocked position or a locked position;
the tubular member can be moved in a first direction and a second direction within the opening in the housing when the first engagement mechanism and the second engagement mechanism are both in the unlocked position;
the tubular member can only be moved in the first direction within the opening in the housing when the first engagement mechanism is in the unlocked position and the second engagement mechanism is in the locked position;
the tubular member can only be moved in the second direction within the opening in the housing when the second engagement mechanism is in the unlocked position and the first engagement mechanism is in the locked position; and
the tubular member cannot be moved in either the first direction or the second direction within the opening in the housing when the first engagement mechanism and the second engagement mechanism are both in the locked position.

2. The apparatus of claim 1 wherein the tubular member is a two-piece tubular member comprising a first piece and a second piece, wherein the second piece is configured to secure to the first piece along a longitudinal axis.

3. The apparatus of claim 1 or claim 2 wherein the tubular member comprises:
a first end, a second end, a first set of serrated teeth and a second set of serrated teeth;
the first set of serrated teeth comprises angled surfaces angled toward the first end; and
the second set of serrated teeth comprises angled surfaces angled toward the second end.

4. The apparatus of claim 3 wherein:
the first engagement mechanism comprises a first spring-loaded engagement member configured to engage the first set of serrated teeth; and
the second engagement mechanism comprises a second spring-loaded engagement member configured to engage the second set of serrated teeth.

5. The apparatus of claim 4 wherein:
the first engagement mechanism comprises a first push button configured to rotate a first annular cam such that the first engagement mechanism can be moved from the locked position to the unlocked position and from the unlocked position to the locked position by depressing the first push button; and
the second engagement mechanism comprises a second push button configured to rotate a second annular cam such that the second engagement mechanism can be moved from the locked position to the unlocked position and from the unlocked position to the locked position by depressing the second push button.

6. The apparatus of claim 5 wherein:
the first engagement mechanism further comprises:
a first barrel with a first plurality of grooves; and
a first rotating cam with a first plurality of ridges, wherein the first plurality of ridges is slidably engaged with the first plurality of grooves; and
the second engagement mechanism further comprises:
a second barrel with a second plurality of grooves; and
a second rotating cam with a second plurality of ridges, wherein the second plurality of ridges are slidably engaged with the second plurality of grooves.

7. The apparatus of claim 4 wherein:
the first engagement mechanism comprises a first rotatable cap configured to rotate such that the first engagement mechanism can be moved from the locked position to the unlocked position and from the unlocked position to the locked position; and
the second engagement mechanism comprises a second rotatable cap configured to rotate such that the second engagement mechanism can be moved from the locked position to the unlocked position and from the unlocked position to the locked position.

8. The apparatus of any one of claims 5-7 wherein:
the first engagement mechanism comprises:
a first spring engaging the first spring-loaded engagement member; and
a second spring engaging the first spring-loaded engagement member; and
the second engagement mechanism comprises:
a third spring engaging the second spring-loaded engagement member; and
a fourth spring engaging the second spring-loaded engagement member.

9. The apparatus of claim 8 wherein:
the first spring exerts a force on the first spring-loaded engagement member toward the first set of serrated teeth;
the second spring exerts a force on the first spring-loaded engagement member away from the first set of serrated teeth;
the third spring exerts a force on the second spring-loaded engagement member toward the second set of serrated teeth; and
the fourth spring exerts a force on the second spring-loaded engagement member away from the second set of serrated teeth.

10. The apparatus of any one of claims 3-9 wherein:
the first spring-loaded engagement member engages the first set of serrated teeth when the first engagement mechanism is in the locked position;
the first spring-loaded engagement member does not engage the first set of serrated teeth when the first engagement mechanism is in the unlocked position;
the second spring-loaded engagement member engages the second set of serrated teeth when the second engagement mechanism is in the unlocked position; and
the second spring-loaded engagement member does not engage the second set of serrated teeth when the second engagement mechanism is in the unlocked position.

11. The apparatus of claim 9 or claim 10 wherein:
movement of the tubular member within the opening in the housing in a first direction compresses the first spring when the first engagement mechanism is in the locked position; and
movement of the tubular member within the opening in the housing in a second direction compresses the third spring when the second engagement mechanism is in the locked position.

12. The apparatus of any one of claims 8-11 wherein:
the first engagement mechanism comprises a first locking member positioned between the first spring and the first rotatable cap; and
the second engagement mechanism comprises a second locking member positioned between the third spring and the second rotatable cap.

13. The apparatus of claim 12 wherein:
the first engagement mechanism can be moved from the unlocked position to the locked position by compressing the first spring with the first locking member and rotating the first rotatable cap; and
the second engagement mechanism can be moved from the unlocked position to the locked position by compressing the third spring with the second locking member and rotating the second rotatable cap.

14. The apparatus of claim 12 or claim 13 wherein:
the first rotatable cap comprises a first aperture;
the second rotatable cap comprises a second aperture;
the first locking member is configured to extend through the first aperture when the first engagement mechanism is in the unlocked position;
the second locking member is configured to extend through the second aperture when the second engagement mechanism is in the unlocked position;
the first locking member is configured to be contained within the first rotatable cap when the first engagement mechanism is in the locked position; and
the second locking member is configured to be contained within the second rotatable cap when the second engagement mechanism is in the locked position.

15. The apparatus of any one of claims 1-14 further comprising an endotracheal tube stabilization device, wherein the endotracheal tube stabilization device comprises:
a first pad coupled to a first extension; and
a second pad coupled to a second extension.

16. The apparatus of claim 15 wherein the first pad and the second pad comprise an adhesive.

17. The apparatus of any one of claims 15-16 wherein the housing comprises a slot configured to receive the endotracheal tube stabilization device.

18. The apparatus of any one of claims 8-17 wherein:
the first spring-loaded engagement member comprises a first planar member configured to engage the first spring and the second spring; and
the second spring-loaded engagement member comprises a second planar member configured to engage the third spring and the fourth spring.

19. The apparatus of claim 18 wherein:
the first spring-loaded engagement member comprises a first extension extending from the first planar member;
the first extension comprises a first angled end configured to engage the first set of serrated teeth;
the second spring-loaded engagement member comprises a second extension extending from second the planar member; and
the second extension comprises a second angled end configured to engage the second set of serrated teeth.

20. The apparatus of claim 19 wherein:
the first extension extends through the second spring; and
the second extension extends through the fourth spring.

21. The apparatus of any one of claims 1-20 wherein the tubular member is configured to receive an oro-endotracheal tube.

22. The apparatus of any one of claims 1-20 wherein the tubular member is configured to receive a naso-endotracheal tube.

23. A method of adjusting an endotracheal tube, the method comprising:
coupling an apparatus to an endotracheal tube stabilization device, wherein the apparatus comprises:
a housing comprising an opening configured to receive the tubular member and the endotracheal tube;
a first engagement mechanism coupled to the housing, wherein the first engagement mechanism is configured to be placed in either an unlocked position or a locked position;
a second engagement mechanism coupled to the housing, wherein the second engagement mechanism is configured to be placed in either an unlocked position or a locked position;
coupling a tubular member to an endotracheal tube;
inserting the tubular member and the endotracheal tube into the opening of the housing;
moving the first engagement mechanism from the unlocked position to the locked position;
adjusting the tubular member and the endotracheal tube within the opening in the housing; and
moving the second engagement mechanism from the unlocked position to the locked position, such that the tubular member and the endotracheal tube are restricted from further movement within the opening in the housing.

24. The method of claim 1 wherein adjusting the tubular member and the endotracheal tube within the opening in the housing provides a precise endotracheal tube position adjustment within an airway of a patient.

25. The method of claim 24 wherein the endotracheal tube is inserted into the airway of the patient via an oral passage.

26. The method of claim 24 wherein the endotracheal tube is inserted into the airway of the patient via a nasal passage.

27. The method of claim 24 wherein moving the first engagement mechanism from the unlocked position to the locked position and moving the second engagement mechanism from the unlocked position to the locked position holds the endotracheal tube in a stable position in the airway of the patient.

28. The method of claim 23 wherein:
the first engagement mechanism comprises a first rotatable cap, a first spring, a second spring and a first locking member;
the second engagement mechanism comprises a second rotatable cap, a third spring, a fourth spring, and a second locking member;
moving the first engagement mechanism from the unlocked position to the locked position comprises:
compressing the first spring by depressing the first locking member within the first rotatable cap; and
rotating the first rotatable cap; and
moving the second engagement mechanism from the unlocked position to the locked position comprises:
compressing the third spring by depressing the second locking member within the second rotatable cap; and
rotating the second rotatable cap.

29. The method of claim 23 wherein:
the first engagement mechanism comprises a first push button, a first spring, a second spring and a first locking member;
the second engagement mechanism comprises a second push button, a third spring, a fourth spring, and a second locking member;
moving the first engagement mechanism from the unlocked position to the locked position comprises compressing the first spring by depressing the first push button; and
moving the second engagement mechanism from the unlocked position to the locked position comprises compressing the third spring by depressing the second push button.

30. The method of claim 5 wherein:
the first engagement mechanism further comprises:
a first barrel with a first plurality of grooves; and
a first rotating cam with a first plurality of ridges, wherein the first plurality of ridges are slidably engaged with the first plurality of grooves;
the second engagement mechanism further comprises:
a second barrel with a second plurality of grooves; and
a second rotating cam with a second plurality of ridges, wherein the second plurality of ridges are slidably engaged with the second plurality of grooves.

31. The method of any one of claims 23-30 wherein:
the tubular member comprises a first set of serrated teeth and a second set of serrated teeth;
the first engagement mechanism comprises a first spring-loaded engagement member;
the second engagement mechanism comprises a second spring-loaded engagement member;
moving the first engagement mechanism from the unlocked position to the locked position causes the first spring-loaded member to engage the first set of serrated teeth; and
moving the second engagement mechanism from the unlocked position to the locked position causes the second spring-loaded member to engage the second set of serrated teeth.

32. The method of claim 31 wherein adjusting the tubular member and the endotracheal tube in a first direction within the opening in the housing provides an audible feedback to a user when the first spring-loaded engagement member is engaged with the first set of serrated teeth.

33. The method of claim 31 or claim 32 wherein adjusting the tubular member and the endotracheal tube in a second direction within the opening in the housing provides an audible feedback to a user when the second spring-loaded engagement member is engaged with the second set of serrated teeth.

34. The method of any one of claims 31-33 wherein adjusting the tubular member and the endotracheal tube in a first direction within the opening in the housing provides a haptic feedback to a user when the first spring-loaded engagement member is engaged with the first set of serrated teeth.

35. The method of any one of claims 31-34 wherein adjusting the tubular member and the endotracheal tube in a second direction within the opening in the housing provides a haptic feedback to a user when the second spring-loaded engagement member is engaged with the second set of serrated teeth.
